(19) United States
(12) Patent Application Publication
(10) Pub. No.: US 2013/0150302 A1
(43) Pub. Date: Jun. 13, 2013

(54) CONJUGATED BLOOD COAGULATION FACTOR VIII

(71) Applicant: William Henry, Haddenham (GB)

(72) Inventor: William Henry, Haddenham (GB)

(73) Assignee: CANTAB BIOPHARMACEUTICALS PATENTS LIMITED, Valletta (MT)

(21) Appl. No.: 13/643,287

(22) PCT Filed: Apr. 28, 2011
(86) PCT No.: PCT/GB2011/000662

§ 371 Date: Feb. 1, 2013

(30) Foreign Application Priority Data
Apr. 30, 2010 (GB) 1007357.5

(51) Int. Cl. C07K 14/755; A61K 45/06; A61K 38/37

(52) U.S. Cl. CPC C07K 14/755; A61K 38/37; A61K 45/06
USPC 514/14.1; 530/383

(57) ABSTRACT

The present invention provides a biocompatible polymer conjugated to FVIII via one or more cysteine residues, suitably via a linker across a reduced disulphide bond in FVIII, and pharmaceutical compositions comprising such conjugated forms of FVIII.

The present invention relates to conjugated forms of the blood coagulation Factor VIII.

Factor VIII (FVIII) is an essential blood clotting factor also known as anti-haemophilic factor (AHF). In humans, Factor VIII is encoded by the F8 gene. Defects in this gene results in haemophilia A, a well known recessive X-linked coagulation disorder effecting approximately 1 in 5,000 males.

The X-linked F8 gene encodes a polypeptide of 2351 amino acids from 26 exons which after signal peptide cleavage renders a mature FVIII molecule of 2332 amino acids (Wang et al. Int. J. Pharmaceutics, 259: 1-15 (2003)). FVIII has been found to be synthesized and released into the bloodstream by the vascular, glomerular, and tubular endothelium, and the sinusoidal cells of the liver though there is still considerable ambiguity as to what the primary site of release in humans is. The FVIII molecule is organised into six protein domains; NH₂-A1-A2-B-A3-C1-C2-COOH. The mature molecule contains a number of post-translational modifications including N-linked and O-linked glycosylation, sulphonation and disulphide bond formation. FVIII contains a total of 23 cysteine residues, 16 of these form 8 disulphide bonds in the A and C domains of the protein (McMullen et al. Protein Science, 4: 740-746 (1995)). Due to the post-translational modification of the protein, its circulation molecular weight can be up to 330 kDa depending on the level and type of glycosylation. FVIII is also proteolytically processes so that the circulating species is a heterodimer composed of a heavy chain (A1-A2-B) and light chain (A3-C1-C2). When FVIII is secreted into the circulation it binds to von Willebrand Factor (vWF) in a non-covalent manner. The binding of the two molecules involves the A3 and C2 domains of the light chain of FVIII (Lacroix-Desmazes et al. Blood, 112: 240-249 (2008)). Binding to vWF increases the stability and circulating half-life of FVIII. Although binding to vWF increases the circulating half-life of FVIII, its native half life is 15-19 hours.

Factor VIII is an essential cofactor participating in the intrinsic blood coagulation pathway.

Its role in the coagulation cascade is to act as a “nucleation template” to organise the components of the FXase complex in the correct spatial orientation on the surface of activated platelets (Shen et al. Blood, 111: 1240-1247 (2008)). FVIII is initially activated by thrombin (Factor IIa) or FXa and it then dissociates from vWF in the form of FVIIIa. FVIIIa then binds to activated platelets at the site of vascular injury and binds FIXa through a A2 and A3 mediated interaction. The binding of FIXa to FVIII in the presence of Ca²⁺ on the platelet surface increases the proteolytic activity of FIXa by approximately 200.000-fold. This complex then activates FX to FXa. Factor Xa, with its cofactor Factor Va, then activates more thrombin. Thrombin in turn cleaves fibrinogen into fibrin which then polymerizes and crosslinks (using Factor XIII) into a fibrin blood clot.

No longer protected by vWF, activated FVIII is proteolytically inactivated in the process (most prominently by activated Protein C and Factor IXa) and quickly clears from the blood stream.

An approach to PEGylation of proteins has been developed by PolyTherics Ltd and is known as TheraPEG™ in which a PEG polymer is attached to the protein of interest via a reduced disulphide bond of a pair of cysteine residues in the protein (WO 2005/007197). The technique has been used to prepare a PEGylated version of Factor IX free of contamination from Factor FIXa (WO 2009/130602).

However, with regard to using this same technology for the PEGylation of FVIII, it was not considered to be trivial or routine.

From the point of view of activity of FVIII with respect to FIX, certain key differences exist which means that conjugation of the protein with a biocompatible polymer is not a straightforward step to take.

For example, FIXa is a serine protease, whereas FVIII has no enzymatic activity. FIX once activated needs only to form an association with its cofactor, which happens to be FVIII, to participate in the coagulation cascade.

In contrast, FVIII is a cofactor and forms a “template” on which other clotting factors (including FIXa) assemble therefore increasing their catalytic activity.

To perform its function FVIII must be able to bind to FX, FXa, FIXa and phospholipids. Also, for FVIII to be stable in the circulation it must be able to bind to von Willebrand Factor. Hence, PEGylation of this protein at disulphides on cysteine residues could sterically hinder these interactions as there are disulphides located in all of the domains of FVIII that carry out intermolecular interactions.

Therefore, PEGylation of factor FVIII presents several unique and different challenges which are distinct and different to that of FIX.

The fact that PEGylation of FIX employing TheraPEG™ technology was successful is no guide to the success or otherwise of PEGylated FVIII prepared using the same approach as it is a structurally and functionally different protein.

It has been discovered that the manufacture of Factor VIII (herein referred to as FVIII) may be enhanced by conjugating FVIII to one or more biocompatible polymers. The enhanced manufacturing properties also include the ability to produce high purity FVIII conjugates.

According to a first aspect of the invention there is provided a biocompatible polymer conjugated to FVIII via one or more cysteine residues.

The biocompatible polymer may be selected from the group consisting of polyethylene glycol (PEG), poly-phosphatidyl choline (PC), polypropylene glycol (PPG), copolymers of ethylene glycol and propylene glycol, polyethylene oxide (PEO), polyoxyethylated polyol, polyolefinic alcohol, polyhydroxyalkylmethacrylate, polysaccharides, poly α-hydroxy acid, polyvinyl alcohol, polyphosphosphasphazene, poly N-acryloylmorpholine, polyalkyene oxide polymers, polymaleic acid, poly DL-alanine, carboxymethylcellulose, dextran, starch or starch derivatives, hyaluronic acid chitin, polymethacrylates, polysialic acid (PSA), polyhydroxy alkanoates, poly amino acids and combinations thereof. The biocompatible polymer may have a linear or branched structure.

In a further embodiment, the biocompatible polymer is a protein selected from, but not limited to, the group consisting of FVII, albumin, transferrin, immunoglobulins including monoclonal antibodies, antibody fragments for example; single-domain antibodies, V_{L}, V_{H}, Fab, F(ab′)₂, Fab′, Fab3, scFv, di-scFv, sdAb, Fc and combinations thereof.

One or more biocompatible polymers may be conjugated to each FVIII molecule if desired via one or more cysteine residues. A free cysteine residue is the result of reducing a cystine disulphide bond. The biocompatible polymer of the invention may be conjugated to FVIII via one or more reduced cysteine disulphide bonds. The conjugation may be by means of a linker group bridging the sulphur residues of two cysteine residues that formed a disulphide bond in FVIII. The disulphide bond may therefore be a native disulphide bond or a recombinantly introduced disulphide bond.

The PEG molecule may be of any suitable molecular weight, for example from 5 to 100 kDa, 10 to 500 kDa. suitably 5 to 30 kDa or 10 to 30 kDa Some suitable molecular weights include 10, 20, or 30 kDa.

Suitably, the biocompatible polymer moiety of the FVIII conjugate may be bound to two cysteine residues, which form a disulphide bond in FVIII. Therefore, the PEG containing linker bridges the disulphide bond. Examples of such conjugation procedures are described in WO 2005/007197, WO 2009/047500 and WO 2010/010324.

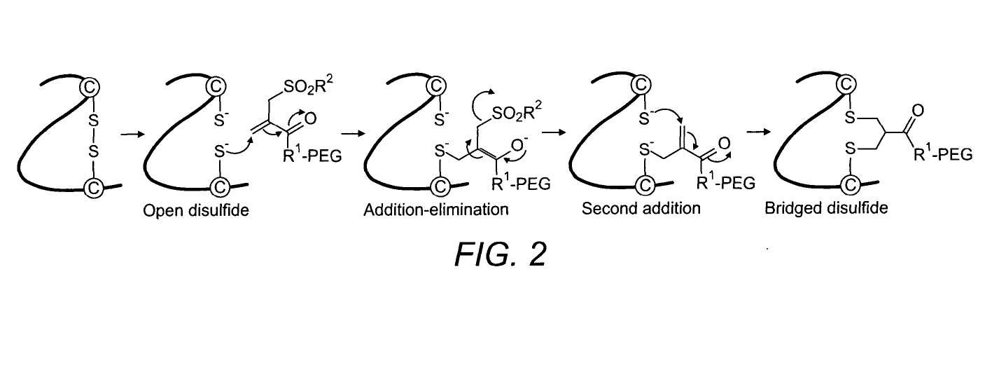

In one embodiment of the invention, a biocompatible polymer can be conjugated to FVIII according to the scheme set out in FIG. 2. In FIG. 2, a group R1 is shown between the biocompatible polymer and the linker group spanning the sulphur atoms of the disulphide bond on the FVIII molecule.

R1 represents a substituent group which can be a direct bond, an alkylene group (preferably a C₁₋₁₀ alkylene group), or an optionally-substituted aryl or heteroaryl group; wherein the aryl groups include phenyl, benzoyl and naphthyl groups; wherein suitable heteroaryl groups include pyridine, pyrrole, furan, pyran, imidazole, pyrazole, oxazole, pyridazine, pyrimidine and purine; wherein linkage to the polymer may be by way of a hydrolytically labile bond, or by a nonlabile bond.

Particular substituents which may be present on the optionally substituted aryl or heteroaryl group include for example one or more of the same or different substituents selected from —CN, —NO₂, —CO₂R, —COH, —CH₂OH, —COR, —OR, —OCOR, —OCO₂R, —SR, —SOR, —SO₂R, —NHCOR, —NRCOR, —NHCO₂R, —NR′CO₂R, —NO, —NHOH, —NR′OH, —C═N—NHCOR, —C═N—NR′COR, —N⁺R₃, —N⁺H₃, —N⁺HR₂, —N⁺H₂R, halogen, for example fluorine or chlorine, —C≡CR, —C═CR₂ and ¹³C═CHR, in which each R or R′ independently represents a hydrogen atom or an alkyl (preferably C₁₋₆) or an aryl (preferably phenyl) group. The presence of electron withdrawing substituents is especially preferred. In one embodiment, the optionally-substituted aryl or heteroaryl group in R1 includes aryl or heteroaryl groups substituted by an amide (NHCO) group which connects to the R1 unit to the biocompatible polymer.

The linker group between the two sulphur atoms of the original disulphide bond between the cysteine residues of FVIII may therefore comprise a 3-carbon bridge. In one embodiment, the linker group between the two sulphur atoms of the original disulphide bond between the cysteine residues of FVIII is (CH₂)₂CHC(O)—.

In one embodiment of the invention, the biocompatible polymer may be conjugated as described above wherein the composition comprising FVIII conjugated to a biocompatible polymer has the structure:

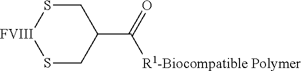

In the broadest sense of the invention, the reagent may be represented as:

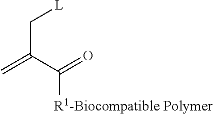

Where R1 is as defined above and L is a leaving group.

L may represent —SR, —SO₂R, —OSO₂R, —N⁺R₃, —N⁺HR₂, —N⁺H₂R, halogen (for example, fluorine or chlorine), or —OW, in which each R independently represents a hydrogen atom or an alkyl (for example C₁-C₆ alkyl) or aryl group (for example phenyl) and W represents a substituted aryl group (for example phenyl) containing at least one electron withdrawing substituent.

In one embodiment, where the leaving group L is SO₂R², in which each R2 independently represents a hydrogen atom or an alkyl (for example C₁-C₆ alkyl) or aryl group (for example phenyl), and R1 is as defined above, the conjugation reagent may have the formula

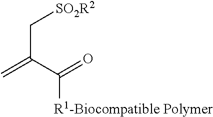

In one embodiment, the biocompatible polymer may be PEG and the leaving group may be —SO₂R², with R2 defined as above, the reagent is as follows:

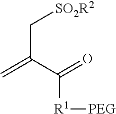

In another embodiment of the invention, the conjugation reagent may be formed from a specific arrangement in which the biocompatible polymer is connected via an amide moiety (CONH), where L is a leaving group as defined above. In other words, R1 is R3-CONH and the reagent has the following formula:

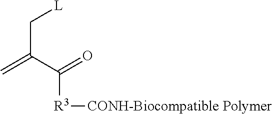

R3 represents a substituent which can be a direct bond, an alkylene group (preferably a C₁₋₁₀ alkylene group), or an optionally-substituted aryl or heteroaryl group; wherein the aryl groups include phenyl, benzoyl and naphthyl groups; wherein suitable heteroaryl groups include pyridine, pyrrole, furan, pyran, imidazole, pyrazole, oxazole, pyridazine, pyrimidine and purine; wherein linkage to the polymer may be by way of a hydrolytically labile bond, or by a nonlabile bond.

Particular substituents which may be present on the optionally substituted aryl or heteroaryl group include for example one or more of the same or different substituents selected from —CN, —NO₂, —CO₂R, —COH, —CH₂OH, —COR, —OR, —OCOR, —OCO₂R, —SR, —SOR, —SO₂R, —NHCOR, —NRCOR, —NHCO₂R, —NR′CO₂R, —NO, —NHOH, —NR′OH, —C═N—NHCOR, —C═N—NR′COR, —N⁺R₃, —N⁺H₃, —N⁺HR₂, —N⁺H₂R, halogen, for example fluorine or chlorine, —C≡CR, —C═CR₂ and ¹³C═CHR, in which each R or R′ independently represents a hydrogen atom or an alkyl (preferably C₁₋₆) or an aryl (preferably phenyl) group. The presence of electron withdrawing substituents is especially preferred.

In embodiments where the moiety CONH is present and where the leaving group L is —SO₂R², where R2 and R3 are as defined above, the reagent is as follows:

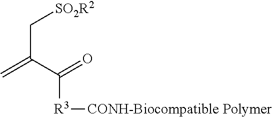

In such embodiments where the optionally-substituted aryl or heteroaryl group in R1, as defined above, of the conjugation reagent includes aryl or heteroaryl groups substituted by an amide (NHCO) group, where R3 is as defined above, the structure of the conjugate protein may be as follows:

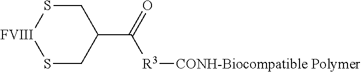

Where the biocompatible polymer is PEG, the conjugation reagent in this embodiment of the invention, where PEG is a polyethylene moiety and L is a leaving group as defined above, is as follows:

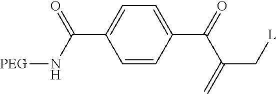

Where the reaction conditions are neutral or slightly basic then the following reagent may be used:

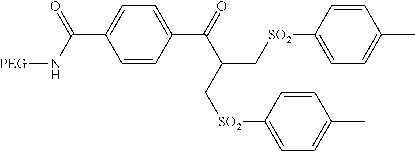

Under more acidic conditions, the above reagent may form the following molecule shown below, PEG mono-sulfone, which is also suitable for use in conjugation reactions as described herein.

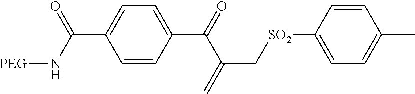

The Factor VIII may be from any suitable source. It may be produced using recombinant DNA technology, or it may be purified from blood plasma. It includes any active fragment or mutein thereof.

As used herein the term “muteins” refers to analogs of a FVIII protein, in which one or more of the amino acid residues of the naturally occurring components of FVIII are replaced by different amino acid residues, or are deleted, or one or more amino acid residues are added to the original sequence of an FVIII, without changing considerably the activity of the resulting products as compared with the original FVIII. These muteins are prepared by known synthesis and/or by site-directed mutagenesis techniques, or any other known technique suitable therefore.

Muteins in accordance with the present invention include proteins encoded by a nucleic acid, such as DNA or RNA, which hybridizes to DNA or RNA, which encodes an FVIII, in accordance with the present invention, under stringent conditions. The term “stringent conditions” refers to hybridization and subsequent washing conditions, which those of ordinary skill in the art conventionally refer to as “stringent” (Ausubel et al., Current Protocols in Molecular Biology, Interscience, N.Y., sections 63 and 6.4 (1987, 1992); Sambrook et al., Molecular Cloning: A Laboratory Manual, Cold Spring Harbor Laboratory Press, Cold Spring Harbor, N.Y. (1989)).

Without limitation, examples of stringent conditions include washing conditions 12-20° C. below the calculated Tm of the hybrid under study in, e.g., 2×SSC and 0.5% SDS for 5 minutes, 2×SSC and 0.1% SDS for 15 minutes; 0.1.times.SSC and 0.5% SDS at 37° C. for 30-60 minutes and then, a 0.1×SSC and 0.5% SDS at 68° C. for 30-60 minutes. Those of ordinary skill in this art understand that stringency conditions also depend on the length of the DNA sequences, oligonucleotide probes (such as 10-40 bases) or mixed oligonucleotide probes. If mixed probes are used, it is preferable to use tetramethyl ammonium chloride (TMAC) instead of SSC.

Any such mutein preferably has a sequence of amino acids sufficiently duplicative of that of an FVIII, such as to have substantially similar, or even better, activity to FVIII.

One characteristic activity of FVIII is its capability of participate in the blood coagulation cascade and assays to detect FVIII activity are described herein. As long as the mutein has substantial FVIII activity, it can be considered to have substantially similar activity to FVIII. Thus, it can be determined whether any given mutein has at least substantially the same activity as FVIII by means of routine experimentation comprising subjecting such a mutein to assays as described herein.

In a preferred embodiment, any such mutein has at least 40% identity or homology with the amino acid sequence of FVIII. More preferably, it has at least 50%, at least 60%, at least 70%, at least 80% or, most preferably, at least 90%, 95% or 99% identity or homology thereto.

Identity reflects a relationship between two or more polypeptide sequences or two or more polynucleotide sequences, determined by comparing the sequences. In general, identity refers to an exact nucleotide to nucleotide or amino acid to amino acid correspondence of the two polynucleotides or two polypeptide sequences, respectively, over the length of the sequences being compared.

For sequences where there is not an exact correspondence, a “percent identity” may be determined. In general, the two sequences to be compared are aligned to give a maximum correlation between the sequences. This may include inserting “gaps” in either one or both sequences, to enhance the degree of alignment. A percent identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity and homology of two or more sequences are well known in the art. Thus for instance, programs available in the Wisconsin Sequence Analysis Package, version 9.1 (Devereux, et al., Nucleic acids Research, 12: 387 (1984)), for example the programs BESTFIT and GAP, may be used to determine the percentage identity between two polynucleotides and the percentage identity and the percentage homology between two polypeptide sequences. BESTFIT uses the “local homology” algorithm of Smith and Waterman (Advances in Applied Mathematics, 2: 482-489 (1981)) and finds the best single region of similarity between two sequences. Other programs for determining identity and/or similarity between sequences are also known in the art, for instance the BLAST family of programs (Atschul et al., J. Molec. Biol. 215: 403 (1990), accessible through the home page of the NCBI at www.ncbi.nlm.nih.gov) and FASTA (Pearson W R, Methods in Enzymology, 183: 63-98 (1990)).

Muteins of FVIII, which can be used in accordance with the present invention include a finite set of substantially corresponding sequences as substitution peptides which can be routinely obtained by one of ordinary skill in the art, without undue experimentation, based on the teachings and guidance presented herein.

Preferred changes for muteins in accordance with the present invention are what are known as “conservative” substitutions. Conservative amino acid substitutions of FVIII may include synonymous amino acids within a group which have sufficiently similar physicochemical properties that substitution between members of the group will preserve the biological function of the molecule. It is clear that insertions and deletions of amino acids may also be made in the above-defined sequences without altering their function, particularly if the insertions or deletions only involve a few amino acids, e.g., under thirty, and preferably under ten, and do not remove or displace amino acids which are critical to a functional conformation, e.g., cysteine residues. Proteins and muteins produced by such deletions and/or insertions come within the scope of the present invention.

Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). Substitutions of this nature are often referred to as “conservative” or “semi-conservative” amino acid substitutions.

Amino acid changes relative to the sequence for the fusion protein of the invention can be made using any suitable technique e.g. by using site-directed mutagenesis.

It should be appreciated that amino acid substitutions or insertions within the scope of the present invention can be made using naturally occurring or non-naturally occurring amino acids. Whether or not natural or synthetic amino acids are used, it is preferred that only L-amino acids are present.

In addition fusion proteins comprising FVIII and another peptide or protein fragment may be also be used provided that the fusion protein retains the activity of FVIII. The term “fusion protein” in this text means, in general terms, one or more proteins joined together by chemical means, including hydrogen bonds or salt bridges, or by peptide bonds through protein synthesis or both.

“Functional derivatives” as used herein cover derivatives of FVIII, and their muteins, which may be prepared from the functional groups which occur as side chains on the residues or are additions to the N- or C-terminal groups, by means known in the art, and are included in the invention as long as they remain pharmaceutically acceptable, i.e. they do not destroy the activity of the protein which is substantially similar to the activity of FVIII, and do not confer toxic properties on compositions containing it.

These derivatives may, for example, include aliphatic esters of the carboxyl groups, amides of the carboxyl groups by reaction with ammonia or with primary or secondary amines, N-acyl derivatives of free amino groups of the amino acid residues formed with acyl moieties (e.g. alkanoyl or carboxylic aroyl groups) or O-acyl derivatives of free hydroxyl groups (for example that of seryl or threonyl residues) formed with acyl moieties, including for example glycosylation of available hydroxyl residues.

An “active fragment of FVIII” according to the present invention may be a fragment of FVIII or a mutein as defined herein. The term fragment refers to any subset of the molecule, that is, a shorter peptide that retains the desired biological activity. Fragments may readily be prepared by removing amino acids from either end of the FVIII molecule and testing the resultant fragment for its properties as described herein. Proteases for removing one amino acid at a time from either the N-terminal or the C-terminal of a polypeptide are known, and so determining fragments, which retain the desired biological activity, involves only routine experimentation.

As active fractions of an FVIII, muteins and active fragments thereof, the present invention further covers any fragment or precursors of the polypeptide chain of the protein molecule alone or together with associated molecules or residues linked thereto, e.g., sugar or phosphate residues, or aggregates of the protein molecule or the sugar residues by themselves, provided said fraction has substantially similar activity to FVIII.

The term “salts” herein refers to both salts of carboxyl groups and to acid addition salts of amino groups of the FVIII molecule or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of FVIII as described herein.

As used herein, the terms “Factor VIII conjugate” or “FVIII conjugate” refers to Factor VIII that has been modified to include a biocompatible polymer moiety that results in an improved pharmacokinetic profile as compared to the unmodified Factor VIII. The improvement in the pharmacokinetic profile may be observed as an improvement in one or more of the following parameters: potency, stability, area under the curve, circulating half-life and immunogenicity or cross-reactivity.

Compared to unmodified FVIII, the FVIII conjugates of the invention may show an improvement in one or more parameters of the pharmacokinetic profile, including area under the curve (AUC), Cmax, clearance (CL), half-life, plasma residence time and bioavailability as compared to unmodified FVIII.

The “area under the curve” or “AUC”, as used herein in the context of administering a peptide drug to a patient, is defined as total area under the curve that describes the concentration of drug in systemic circulation in the patient as a function of time from zero to infinity. As used herein the term “clearance” or “renal clearance” is defined as the volume of plasma that contains the amount of drug excreted per minute.

As used herein the term “half-life” or “t½”, in the context of administering a peptide drug to a patient, is defined as the time required for plasma concentration of a drug in a patient to be reduced by one half. There may be more than one half-life associated with the peptide drug depending on multiple clearance mechanisms, redistribution, and other mechanisms well known in the art. Usually, alpha and beta half-lives are defined such that the alpha phase is associated with redistribution, and the beta phase is associated with clearance. However, with protein drugs that are, for the most part, confined to the bloodstream, there can be at least two clearance half-lives. The precise impact of PEGylation on alpha phase and beta phase half-lives will vary depending upon the size and other parameters, as is well known in the art. Further explanation of “half-life” is found in Pharmaceutical Biotechnology (1997, DFA Crommelin and RD Sindelar, eds., Harwood Publishers, Amsterdam, pp 101-120).

As used herein the term “residence time”, in the context of administering a peptide drug to a patient, is defined as the average time that drug stays in the body of the patient after dosing.

As used herein the term “immunogenicity”, in the context of administering a peptide drug to a patient, is defined as the propensity of that drug to illicit an immune response in the patient after dosing, or after repeat dosing.

As used herein the term “cross-reactivity”, in the context of administering a peptide drug to a patient, is defined as the propensity of that drug to bind antibodies which have been raised by the patient after dosing, or repeat dosing, of the un-conjugated drug.

FVIII conjugates may provide therapeutic benefits, for example, when compared to unconjugated FVIII. Such therapeutic benefits include, but are not limited to, increased circulation half-life, decreased immunogenicity, decreased cross-reactivity, higher activity, lower dosing requirements, and allowing for alternative routes of administration (e.g., subcutaneously).

According to the present invention, the conjugation of FVIII with a biocompatible polymer enhances the utility of FVIII in pharmaceutical compositions. Moreover, the biocompatible moiety may protect FVIII from degradation and antibody response. The FVIII conjugates may have a prolonged circulating half-life, which results in a dose-sparing effect and less frequent administration.

Factor VIII can be PEGylated using PolyTherics Ltd TheraPEG™ technology for conjugation of PEG to a disulfide bond. Batches of mono-PEGylated Factor VIII (FVIII) can be prepared using for example 10, 20 and 30 kDa PEG reagents.

The coagulation activity of the PEGylated FVIII can be tested in vitro using a clotting assay.

Factor VIII has been commercially available for decades, and can be purified from donor plasma as a blood product or produced through recombinant DNA technology.

There are several different types of polyethylene glycol polymers that will form conjugates with FVIII. There are linear PEG polymers that contain a single polyethylene glycol chain, and there are branched or multi-arm PEG polymers. Branched polyethylene glycol contains 2 or more separate linear PEG chains bound together through a unifying group. For example, two PEG polymers may be bound together by a lysine residue. One linear PEG chain is bound to the α-amino group, while the other PEG chain is bound to the ε-amino group. The remaining carboxyl group of the lysine core is left available for covalent attachment to a protein. Both linear and branched polyethylene glycol polymers are commercially available in a range of molecular weights.

In one aspect of the invention, a FVIII-PEG conjugate contains one or more linear polyethylene glycol polymers bound to FVIII, in which each PEG has a molecular weight between about 2 kDa to about 100 kDa. In another aspect of the invention, a FVIII-PEG conjugate contains one or more linear polyethylene glycol polymers bound to FVIII, wherein each linear PEG has a molecular weight between about 5 kDa to about 40 kDa. In certain embodiments, each linear PEG has a molecular weight between about 10 kDa to about 30 kDa. In certain embodiments, each linear PEG has a molecular weight that is about 20 kDa. In certain embodiments, each linear PEG has a molecular weight that is less than about 10 kDa.

In particular embodiments, where the FVIII-PEG conjugate contains more than one linear PEG polymers bound to FVIII, for example two, three, or up to eight linear PEG polymers bound to FVIII. In some embodiments, the FVIII-PEG conjugates contain multiple linear PEG polymers, where each linear PEG has a molecular weight of about 10-30 kDa.

A FVIII-PEG conjugate of this invention may contain one or more branched PEG polymers bound to FVIII, wherein each branched PEG has a molecular weight between about 2 kDa to about 100 kDa. In another aspect of the invention, a FVIII-PEG conjugate contains one or more branched polyethylene glycol polymers bound to FVIII, wherein each branched PEG has a molecular weight between about 5 kDa to about 40 kDa. In certain embodiments, each branched PEG has a molecular weight between about 5 kDa to about 30 kDa. In certain embodiments, each branched PEG has a molecular weight that is about 20 kDa. In certain embodiments, each branched PEG has a molecular weight that is less than about 10 kDa. In particular embodiments, where the FVIII-PEG conjugate contains more than one branched PEG polymers bound to FVIII, for example two, three, or up to eight branched PEG polymers bound to FVIII. In a some embodiments, the FVIII-PEG conjugates contains up to eight branched PEG polymers, where each branched PEG has a molecular weight of about 10-30 kDa.

The FVIII-PEG conjugates may be purified by chromatographic methods known in the art, including, but not limited to ion exchange chromatography and size exclusion chromatography, affinity chromatography, precipitation and membrane-based separations.

As discussed above, PolyTherics has developed a technology, known as TheraPEG™ that can exploit the selective chemistry of naturally occurring sulphur atoms in proteins for site-specific PEGylation. The technology can also be applied to proteins and peptides where novel sulphur-containing groups have been introduced by recombinant or other means. PolyTherics has shown that disulphide bonds can be made more stable by the addition of a PEG-linked carbon bridge and that it is possible to make such a modification to disulphide bonds in proteins while retaining tertiary structure and maintaining protein function. This has made it possible for the first time to exploit the conjugating thiol selectivity of the two sulphur atoms that make up a disulphide bond to conjugate biocompatible polymers to a protein of interest site-specifically in either native or selectively engineered proteins. One example, of this approach is to use the technology to add PEG moieties to a FVIII protein (or to “PEGylate” the FVIII protein).

The disulphide-bridging conjugation reagent is a latently cross-conjugated system capable of undergoing interactive Michael and retro-Michael reactions. This enables the two free thiols generated by the reduction of a native disulphide group to re-anneal across a 3 carbon bridge that linked the two sulphur groups of the original disulphide bond (For example see FIG. 2 for a schematic representation of the conjugation reaction to add a PEG moiety). The conjugation reagent may be described as a “PEGylation” reagent when it comprises PEG as the biocompatible polymer used to PEGylate the FVIII protein.

Mechanistically, a conjugated double bond in the conjugation reagent is required to initiate a sequence of addition reactions. Once thiolate addition occurs, elimination of the remaining sulphinic acid moiety becomes possible. This generates another conjugated double bond for the addition of a second thiolate anion and the formation of a 3-carbon bridge between the two sulphur atoms. The end result is two very stable thiol-ether bonds either side of the carbon bridge.

According to a second aspect of the invention there is provided a pharmaceutical composition comprising a biocompatible polymer conjugated to FVIII via one or more cysteine residues as defined in relation to the first aspect of the invention.

The pharmaceutical composition of the invention may further comprise a pharmaceutically acceptable diluent, adjuvant or carrier.

Pharmaceutical compositions adapted for oral administration may be presented as discrete units such as capsules, as solutions, syrups or suspensions (in aqueous or non-aqueous liquids; or as edible foams or whips; or as emulsions). Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc. For the preparation of solutions and syrups, excipients which may be used include for example water, polyols and sugars. For the preparation of suspensions oils (e.g. vegetable oils) may be used to provide oil-in-water or water in oil suspensions.

Pharmaceutical compositions adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable compositions wherein the carrier is a liquid, for administration as a nasal spray or as nasal drops, include aqueous or oil solutions of the active ingredient. Pharmaceutical compositions adapted for administration by inhalation include fine particle dusts or mists which may be generated by means of various types of metered dose pressurised aerosols, nebulizers or insufflators.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water, alcohols, polyols, glycerine and vegetable oils, for example. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

In general, the pharmaceutical compositions may contain preserving agents, solubilising agents, stabilising agents, wetting agents, emulsifiers, sweeteners, colourants, odourants, salts (substances of the present invention may themselves be provided in the form of a pharmaceutically acceptable salt), buffers, coating agents or antioxidants. They may also contain therapeutically active agents in addition to the substance of the present invention. The pharmaceutical compositions of the invention may be employed in combination with pharmaceutically acceptable diluents, adjuvants, or carriers. Such excipients may include, but are not limited to, saline, buffered saline (such as phosphate buffered saline), dextrose, liposomes, water, glycerol, ethanol and combinations thereof.

The pharmaceutical compositions may be administered in any effective, convenient manner effective for treating a patients disease including, for instance, administration by oral, intravenous, subcutaneous, intramuscular, intraosseous, intranasal, or routes among others. In therapy or as a prophylactic, the active agent may be administered to an individual as an injectable composition, for example as a sterile aqueous dispersion, preferably isotonic.

For administration to mammals, and particularly humans, it is expected that the daily dosage of the active agent will be from 0.01 mg/kg body weight, typically around 1 mg/kg. The physician in any event will determine the actual dosage which will be most suitable for an individual which will be dependent on factors including the age, weight, sex and response of the individual. The above dosages are exemplary of the average case. There can, of course, be instances where higher or lower dosages are merited, and such are within the scope of this invention.

Dosages of the substance of the present invention can vary between wide limits, depending upon the disease or disorder to be treated, the age and condition of the individual to be treated, etc. and a physician will ultimately determine appropriate dosages to be used.

This dosage may be repeated as often as appropriate. If side effects develop the amount and/or frequency of the dosage can be reduced, in accordance with normal clinical practice. In one embodiment, the pharmaceutical composition may be administered once every one to fourteen days.

According to a third aspect of the invention, there is provided a pharmaceutical composition of the second aspect and another pharmaceutically active agent. The other pharmaceutically active agent may promote or enhance the activity of FVIII, for example another blood coagulation factor.

The pharmaceutical compositions of the invention may be employed alone or in conjunction with other compounds, such as therapeutic compounds or molecules, e.g. anti-inflammatory drugs, analgesics or antibiotics. Such administration with other compounds may be simultaneous, separate or sequential. The components may be prepared in the form of a kit which may comprise instructions as appropriate.

Preferably, the pharmaceutical composition of the invention and the other therapeutic compound are directly administered to a patient in need thereof.

The invention also provides a kit of parts comprising a pharmaceutical composition of invention, and an administration vehicle including, but not limited to, capsules for oral administration, inhalers for lung administration and injectable solutions for intravenous administration.

According to a fourth aspect of the invention, there is provided a method of treatment of a blood clotting disease where the method comprises administration of a composition of the present invention to a patient in need thereof. This aspect of the invention therefore also includes uses of such compositions in said methods.

Blood clotting diseases may be characterised by a loss of function of a blood clotting factor, or the generation of auto-antibodies. Examples of blood clotting diseases includes haemophilia A and acquired haemophilia A.

As used herein, the term “treatment” includes any regime that can benefit a human or a non-human animal. The treatment of “non-human animals” extends to the treatment of domestic animals, including horses and companion animals (e.g. cats and dogs) and farm/agricultural animals including members of the ovine, caprine, porcine, bovine and equine families. The treatment may be in respect of any existing condition or disorder, or may be prophylactic (preventive treatment). The treatment may be of an inherited or an acquired disease. The treatment may be of an acute or chronic condition.

According to a fifth aspect of the invention, there is provided a process for preparing the following conjugate of a biocompatible polymer and FVIII,

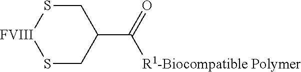

wherein the process comprises:
(a) reduction of a native disulphide bond between two cysteine residues in FVIII, to generate two free thiol groups;
(b) a first thiolate addition reaction between a conjugation-reagent comprising a conjugated double bond and a leaving group;
(c) elimination of the leaving group, generating a conjugated double bond; and
(d) a second thiolate addition reaction, forming a 3-carbon bridge between the two sulphur atoms.

In such a process, the conjugation reagent may have the formula, as described above, of:

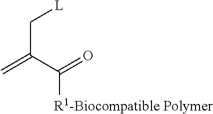

where R1 is as defined above and L is a leaving group as defined above.

Further aspects of this embodiment of the invention are as described above in relation to the various structures of the conjugation reagent.

One example of a conjugation reagent which can be used, with substituents as defined above, in which R1 and R2 are as defined above, is as follows:

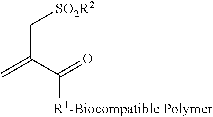

in which the leaving group is a sulfonyl group represented by SO₂R².

Where the biocompatible polymer is PEG, the conjugation reagent, where R2 and R1 are as defined above, can be as follows:

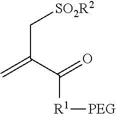

Preferred features for the second and subsequent aspects of the invention are as for the first aspect mutatis mutandis.

The present invention will now be further described with reference to the following Examples which are included for the purposes of reference and are not be construed as being limiting on the claimed invention.

In the present description is made to a number of Figures in which:

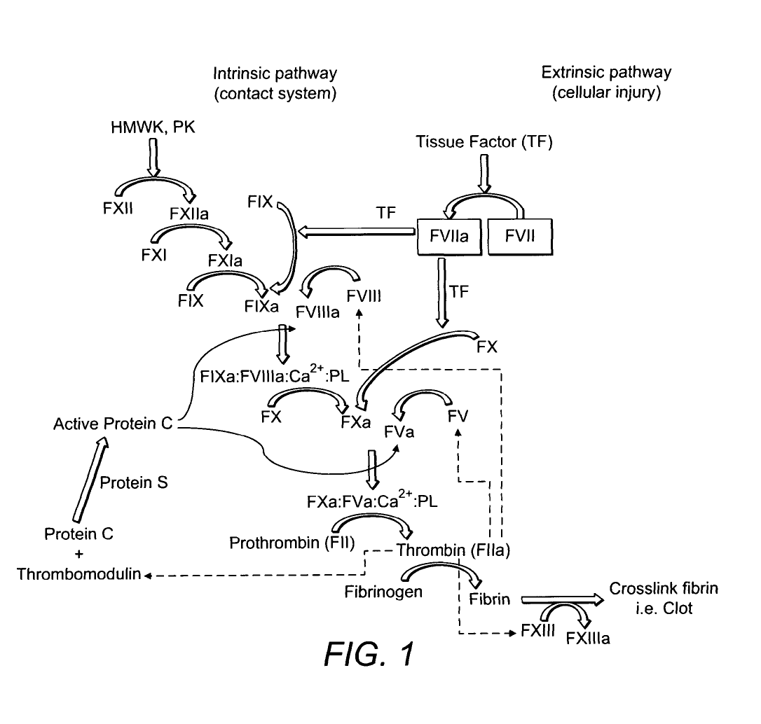

FIG. 1 shows the blood factor coagulation cascade. Abbreviations: HMWK—High Molecular Weight Kininogen; PK—Prekallikrein; PL—Phospholipid.

FIG. 2 shows steps involved in disulfide-specific PEGylation chemistry (from Shaunak et al. in Nat Chem Biol. 2006; 2(6):312-313).

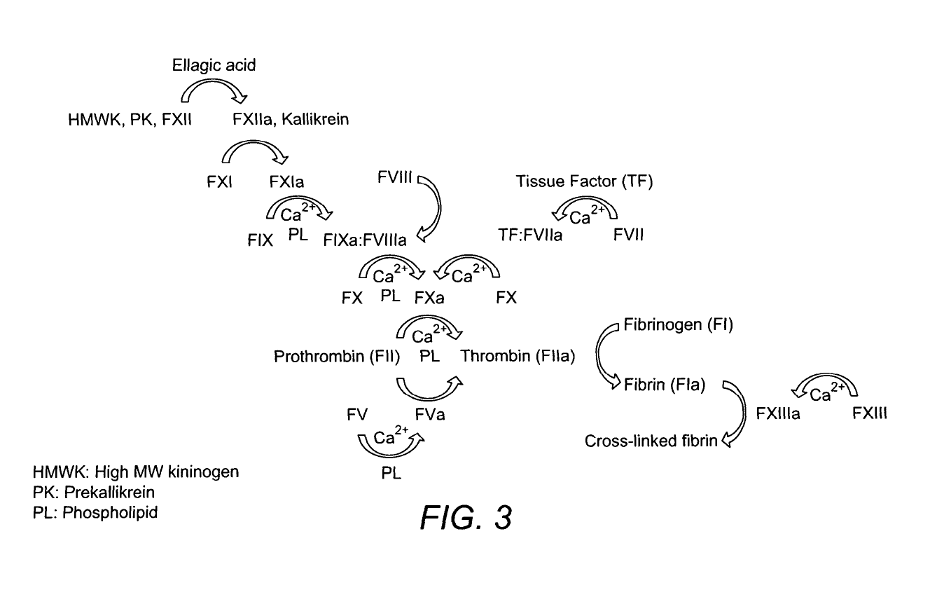

FIG. 3 shows schematic diagram of the steps involved in an aPTT clotting assay. Abbreviations: HMWK—High Molecular Weight Kininogen; PK—Prekallikrein; PL—Phospholipid.

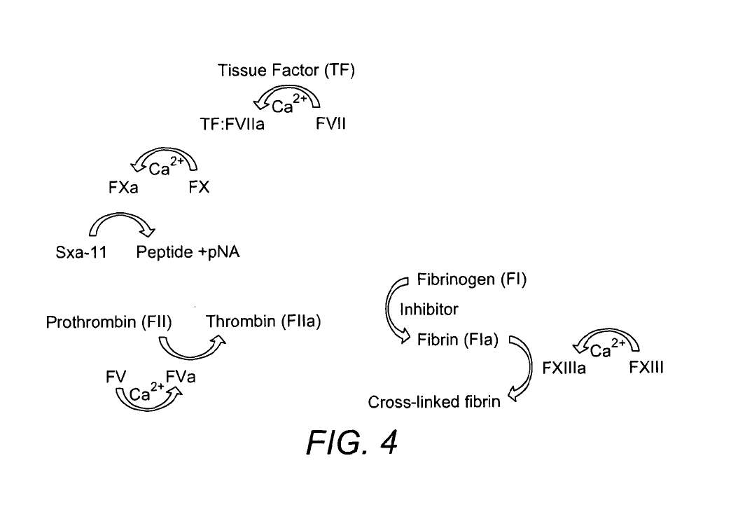

FIG. 4 shows schematic diagram of the steps involved in a chromogenic clotting assay.

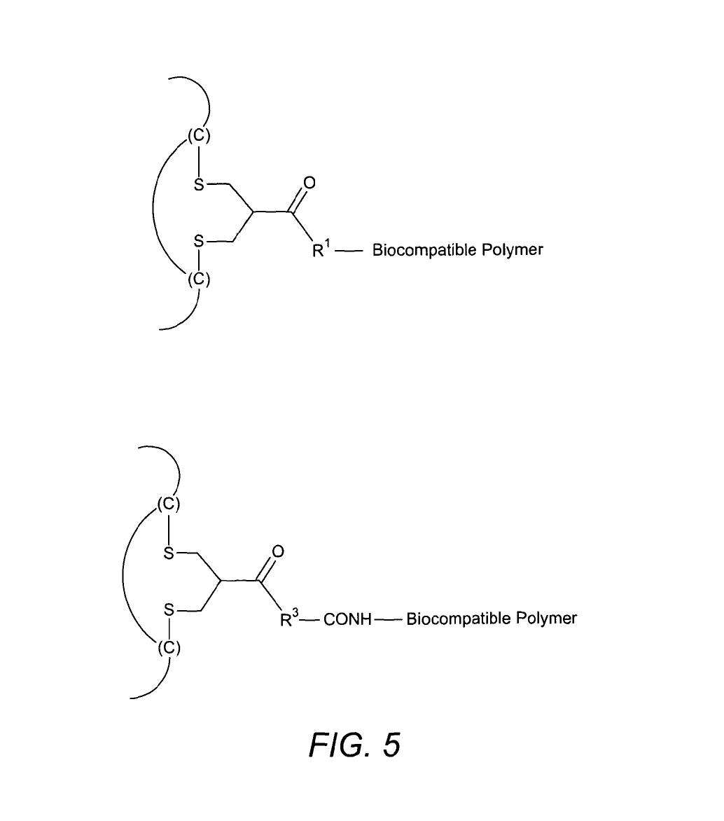

FIG. 5 shows two alternative schematic structures of conjugates of the invention in which FVIII is represented by a black curved line, (C) represents a cysteine residue of FVIII and where FVIII is shown conjugated to a biocompatible polymer by a linker as described herein.

The invention will now be described further with reference to the following Examples which are present for the purposes of illustration only.

### EXAMPLE 1

### Disulphide PEGylation of FVIII

Disulphide PEGylation of human FVIII is carried out according to a modified version of the procedure described by Shaunak et al. in Nat Chem Biol. 2006; 2(6):312-313.

### EXAMPLE 2

### Disulfide Bond Reduction

The TheraPEG™ PEGylation process requires reduction of disulphide bonds. Reduction is performed with an appropriate reducing agent for example dithiothreitol (DTT), 2-mercaptoethanol or tris(2-carboxyethyl) phosphine (TCEP), either in the presence or absence of selenocystamine (SeCys). Concentrations of reducing agents used are for example 0.5-5 mm DTT or a low molar excess of TCEP.

### EXAMPLE 3

### PEGylation of FVIII

Initial assessment of the use of TheraPEG™ for PEGylation of FVIII is carried out in small scale reactions (e.g. 5-20 μg FVIII). This allows identification of conditions that could be used to reproducibly prepare PEGylated FVIII using a PEG reagent. Benzamidine or other excipients can be added to prevent proteolysis or to aid protein stability.

Reactions are scaled up (e.g. 0.2-0.3 mg FVIII) to produce PEGylated FVIII for initial in vitro assessment. A range of PEG-FVIII samples with different PEG molecular weights (e.g. 10, 20 and 30 kDa) and number of conjugated PEG moieties (e.g. 1-8) are produced for in vitro analysis.

The effect of temperature of the PEGylation reactions can be assessed to determine if it effects the conversion of FVIII to PEG-FVIII. However, if initial in vitro assessments indicate that higher temperatures (e.g. 10-30° C.) may have a negative effect on the activity of the PEGylated product then therefore subsequent reactions will be carried out at lower temperatures (e.g. 2-10° C.).

Various purification techniques known in the art may be used for isolation and purification of the PEGylated material. Such techniques include, but are not limited to, ion exchange chromatography, size-exclusion chromatography, affinity chromatography, precipitation or membrane-based separation techniques.

To generate larger quantities of material for further in vitro assessment, reactions are scaled up to for example 1 mg FVIII. Using conditions determined in smaller scale reactions PEGylated variants of FVIII are prepared.

After purification, PEGylated products are analysed by for example SDS-PAGE or SE-HPLC to demonstrate purity and quantified by an appropriate protein assay for example the BCA assay. All of these methods are well known to someone skilled in the art.

### EXAMPLE 4

### Evaluation of In Vitro Activity of PEGylated FVIII

The activity of FVIII and PEGylated FVIII is determined using chromogenic assay and a modified activated partial thromboplastin time coagulation assay.

### Chromogenic Assay

The chromogenic assay (Hypen Biomed catalogue number The chromogenic assay (Hyphen Biomed, catalogue no. 221402) measures the activity of FVIII by formation of a coloured substrate, and does not involve clot formation. See FIG. 3 for a schematic diagram of the steps involved in the chromogenic assay.

When activated by thrombin, Factor VIII:C forms an enzymatic complex with Factor IXa, phospholipids and Calcium, which activates Factor X to Factor Xa. Factor VIII:C is a chromogenic assay for testing the cofactor activity of Factor VIII:C. In presence of a constant amount of Factor IXa, Phospholipids (PLPs) and Calcium, thrombin activated Factor VIII:C forms an enzymatic complex, which activates Factor X, supplied in the assay at a constant concentration and in excess, to Factor Xa. This activity is directly related to the amount of Factor VIII:C, which is the limiting factor in presence of a constant and in excess amount of Factor IXa. Generated Factor Xa is then exactly measured by its activity on a specific Factor Xa chromogenic substrate (SXa-11). Factor Xa cleaves the substrate and releases pNA. The amount of pNA generated is directly proportional to the Factor Xa activity. Finally, there is a direct relationship between the amount of Factor VIII:C in the assayed sample and the Factor Xa activity generated, measured by the amount of pNA released, determined by colour development at 405 nm.

A study comparing Factor VIII activity-based assays using 5 pharmacological factor VIII products was carried out by Butenas et al. (Blood (ASH Annual Meeting Abstracts) 2004 104: Abstract 4012).

### Coagulation Assay

The clotting FVIII test method is a one-stage assay based upon the activated partial thromboplastin time (aPTT). FVIII acts as a cofactor in the presence of Factor IXa, calcium, and phospholipid in the enzymatic conversion of Factor X to Xa. An inverse relationship exists between the time (seconds) it takes for a clot to form and logarithm of the concentration of FVIII.

### Compositions

1. Factor VIII deficent plasma, Helena Biosciences, catalogue number 5193
2. aPTT-ES reagent Helena Biosciences catalogue number 5397
3. Calcium chloride solution, 0.025 mol/L

25 μl of diluted test samples are incubated and 25 μl of FVIII deficient plasma are incubated with 50 ul of pre-warmed aPTT-ES reagent. The activator initiates the contact system. Then, the remaining steps of the intrinsic pathway take place in the presence of phospholipid. After exactly 3 minutes incubation at 37° C. 50 μl of 0.025 mol/L calcium chloride is added and clotting is initiated. The clot times were determined on a Sysmex CA-50 coagulation analyser.

FVIII and PEGylated FVIII activity is determined against the WHO International FVIII standard (NIBSC). Activity levels for unknown samples are interpolated by comparing the clotting times of various dilutions of test material against a standard curve made from a series of dilutions of FVIII standard material of known activity and are reported in International Units per mL (IU/mL). The percentage retained specific clotting activity for PEGylated FVIII is also calculated

In order to establish whether PEGylated FVIII clots at the same rate as unmodified FVIII the clotting time between a 2 to 80% change in scattered light detection is measured and the clot time plotted versus % change in scattered light. The slope of the curves is taken as the rate of the clotting reaction. This is performed by using the same four concentrations of FVIII and PEGylated FVIII.

1. (canceled)

2. (canceled)

3. A Factor VIII-polyethylene glycol conjugate, wherein one or more polyethylene glycol groups are conjugated to FVIII by a linker group bridging the sulphur atoms of two cysteine residues that formed a disulphide bond in FVIII,
wherein the conjugate has the structure: 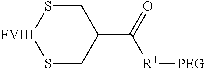
wherein R1 is a substituent which is a direct bond, an alkylene group (preferably a C₁₋₁₀ alkylene group), or an optionally-substituted aryl or heteroaryl group; wherein the aryl groups include phenyl, benzoyl and naphthyl groups; wherein suitable heteroaryl groups include pyridine, pyrrole, furan, pyran, imidazole, pyrazole, oxazole, pyridazine, pyrimidine and purine; wherein linkage to the polymer is by a hydrolytically labile bond, or by a nonlabile bond.

4. (canceled)

5. (canceled)

6. (canceled)

7. (canceled)

8. (canceled)

9. The Factor VIII-polyethylene glycol conjugate of claim 3 wherein the polyethylene glycol has a molecular weight of about 5-100 kDa.

10. A pharmaceutical composition comprising the Factor VIII-polyethylene glycol conjugate of claim 3.

11. The pharmaceutical composition of claim 10, further comprising a pharmaceutically acceptable diluent, adjuvant or carrier.

12. The pharmaceutical composition of claim 11, further comprising another pharmaceutically active agent.

13. The pharmaceutical composition of claim 10, wherein the composition suitable for parenteral administration.

14. The pharmaceutical composition of claim 10, wherein the composition suitable for intradermal, subcutaneous, and intramuscular injections, and intravenous or intraosseous infusions.

15. The pharmaceutical composition of claim 10, wherein the composition is in the form of a solution, suspension or emulsion.

16. The pharmaceutical composition of claim 10, wherein the FVIII conjugate has a longer half-life as compared to unmodified FVIII.

17. The pharmaceutical composition of claim 10, wherein the FVIII conjugate has a higher AUC as compared to unmodified FVIII.

18. The pharmaceutical composition of claim 10, wherein the FVIII conjugate has a higher bioavailability as compared to unmodified FVIII.

19. The pharmaceutical composition of claim 10, wherein the FVIII conjugate has a lower immunogenicity as compared to unmodified FVIII.

20. A method of treatment of a blood clotting disease or trauma comprising administration of the pharmaceutical composition of claim 10.

21. The method of treatment of claim 20, wherein the blood clotting disease is haemophilia A or haemophilia B.

22. A method to reduce the risk of hemarthrosis, hemorrhage, gastrointestinal bleeding and menorrhagia in mammals with haemophilia A, haemophilia B or trauma, comprising administering to a patient in need thereof a pharmaceutical composition comprising the FVIII polyethylene glycolconjugate of claim 10.

23. The method of claim 22, wherein the composition is administered subcutaneously.

24. The method of claim 22, wherein the composition is administered intravenously.

25. The method of claim 22, wherein the composition is administered once every one to fourteen days.

26. A Factor VIII-polyethylene glycol conjugate of claim 3 for use in the treatment of a blood clotting disease characterized by a loss of function of FVIII, or for use in the treatment of trauma.

27. A process for preparing a Factor VIII-polyethylene glycol conjugate wherein the conjugate has the structure: 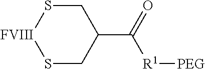
wherein R1 is a substituent which is a direct bond, an alkylene group (preferably a C₁₋₁₀ alkylene group), or an optionally-substituted aryl or heteroaryl group; wherein the aryl groups include phenyl, benzoyl and naphthyl groups; wherein suitable heteroaryl groups include pyridine, pyrrole, furan, pyran, imidazole, pyrazole, oxazole, pyridazine, pyrimidine and purine; wherein linkage to the polymer is by a hydrolytically labile bond, or by a nonlabile bond, and
wherein the process comprises:
(a) reduction of a native disulphide bond between two cysteine residues in FVIII, to generate two free thiol groups;
(b) a first thiolate addition reaction between a conjugation-reagent comprising a conjugated double bond and a leaving group;
(c) elimination of the leaving group, generating a conjugated double bond; and
(d) a second thiolate addition reaction, forming a 3-carbon bridge between the two sulphur atoms.

28. The process of claim 27, wherein the conjugation reagent has the formula 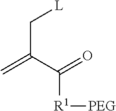
where R1 is a substituent which is a direct bond, an alkylene group (preferably a C₁₋₁₀ alkylene group), or an optionally-substituted aryl or heteroaryl group; wherein the aryl groups include phenyl, benzoyl and naphthyl groups; wherein suitable heteroaryl groups include pyridine, pyrrole, furan, pyran, imidazole, pyrazole, oxazole, pyridazine, pyrimidine and purine; wherein linkage to the polymer is by a hydrolytically labile bond, or by a nonlabile bond, and L is a leaving group.
